(19) United States
(12) Patent Application Publication
(10) Pub. No.: US 2010/0055212 A1
(43) Pub. Date: Mar. 4, 2010

(54) COSMETIC COMPOSITION COMPRISING TISSUE CULTURED PANAX GINSENG C.A. MEYERADVENTITIOUS ROOT ITSELF AND A PREPARING METHOD THEREOF

(76) Inventors: Jong-Wan Choi, Seoul (KR); Bong-Seok Seo, Bucheon-si (KR); Min-Seok Joung, Seoul (KR); Youn-Hee Lee, Seoul (KR); Chang-Min Park, Chungogeonbuk-do (KR)

Correspondence Address: HAMRE, SCHUMANN, MUELLER & LARSON, P.C., P.O. BOX 2902, MINNEAPOLIS, MN, 55402-0902, US

(21) Appl. No.: 12/522,241

(22) PCT Filed: Jan. 17, 2007
(86) PCT No.: PCT/KR07/01058

§ 371 Date: Jul. 6, 2009

(30) Foreign Application Priority Data
Jan. 17, 2007 (KR) 10-2007-0005237

(51) Int. Cl. A61K 8/97; A61Q 19/08

(52) U.S. Cl. 424/728

(57) ABSTRACT

The present invention relates to a cosmetic composition comprising tissue-cultured adventitious roots itself of ginseng, and to a preparing method thereof. The cosmetic composition of the present invention is characterized in that effective ingredients are dip-extracted from the tissue cultured adventitious roots of a ginseng contained in the composition, without any additional process for preparing an extract of the tissue-cultured adventitious roots of a ginseng. In the cosmetic composition of the present invention, the effective ingredients are naturally and continuously dip-extracted from the tissue-cultured adventitious roots itself of a ginseng and maintained, and also allows a user to recognize visually the presence of the adventitious roots of a ginseng, thereby giving trust of the presence of the effective ingredient to the user, and improving user's satisfaction through its natural flavor. Further, the preparing method for the cosmetic composition is a simple and inexpensive process without any additional step, such as extraction, isolation and purification, and further has an advantage that an effective ingredient is dip-extracted from the adventitious roots itself of ginseng while not losing an effective ingredient.

### TECHNICAL FIELD

The present invention relates to a cosmetic composition comprising tissue-cultured adventitious roots itself of a ginseng, and to a preparing method thereof. This application claims priority from Korean Patent Application No. 10-2007-0005237 filed on Jan. 17, 2007 in the Korean Intellectual Property Office, the disclosure of which is incorporated herein by reference in its entirety.

### BACKGROUND ART

Generally, functional cosmetics comprising a plant extract is prepared through multiple steps such as seeding, culturing, harvesting, washing, drying, dipping, aging, and filtering for a cosmetic composition. Therefore, there is a drawback that the process for preparing the cosmetics requires long time and high cost.

It is possible that a large amount of ingredients having a high purity can be obtained in short time in the case of an extraction or supercritical extraction method using a solvent. However, the ingredients extracted by the extraction method have problems in their stability such that they can be precipitated due to light, air, temperature or the like, or their colors can be changed with the passage of time. Accordingly, upon adding the ingredients to a cosmetic composition, there is caused a problem that they may affect the properties of the cosmetic composition.

Since the extracts are typically obtained through dipping or aging for a short time without aging for a sufficient period, they have a problem that a sufficient effect of extraction of effective ingredients is hardly expected. Further, since the effective ingredients cannot be extracted in a large amount without further isolation and/or purification step, cosmetics comprising a high concentration of the effective ingredients is hardly prepared with the extract itself.

A consumer to purchase functional cosmetics cannot recognize visually the presence and contents of an extract containing the effective ingredients, and cannot but purchase and use depending on only labeled ingredients on the package of a cosmetic.

In order to improve the above-described problems, a preparing method for cosmetics comprising a plant itself, not a plant extract, was proposed. However, the method has a drawback that cost is required for sterilizing the plant and a product having uniform quality cannot be achieved due to its different size, harvesting places and periods of the plant added to composition.

A cosmetic composition comprising an undifferentiated callus using a recent tissue culture technique was disclosed in Korean Patent No. 637342. A callus means a cell mass of which function is not determined. Accordingly, a callus has been known to have different types and contents of effective ingredients from those of the differentiated plants.

A korean wild ginseng (“Sansam”), which is an example of a plant containing a large amount of a functional ingredient, belongs to Panax ginseng C. A. Meyer (hereinafter “ginseng”) of the Araliacea family, and grows naturally in a mountain. A korean wild ginseng belongs to a flowering plant and angiosperms, in which a peduncle grows with leafs and a stalk upon budding, and to a semi-shade plant.

A korean wild ginseng has been recorded as a “mysterious herb” in Dongeui Bogam (the integration of traditional Korean medicine). Traditionally, it has been thought to be a celebrated medicinal herb as a rare herb, and as a result, it has been used as a therapeutic agent for various diseases and a nutrient supplement.

The main component of a ginseng is a glycoside. The mixture of glycosides is hydrolyzed with an acid to give a free sugar such as glucose, rhamnose, sucrose and fructose, and aglycone, and to give panaxadiol and panaxatriol, β-sistosterol and oleanolic acid as a saponin glycoside. Panacene, which is a sesquiterpenoid compound, is an aromatic component. Further, it has been known that other components, such as β-sistosterol, stigumasterol; alkaloid; flavonoids such as kaempherol, trifolime, and panasenoid; vitamins such as B complex, nicotinic acid, pantothenic acid, folic acid and biotin; trace elements such as manganese, copper, vanadium, cobalt, arsenic, germanium, phosphorus, aluminum and nickel; germanium cell enzyme; and enzymes such as amylase, and phenolase, are contained.

According to the results from clinical research, it has been found that a ginseng has an efficacy such as antidiabetic effect, anticancer effect, cardio-stimulation, regulation of blood pressure, stimulation of liver function, stimulation of stomach, stress relief and restorative activity, stamina enhancement, stimulation of brain function, anti-aging activity, radiation protection, effect on anemia and hematopoiesis, immunity enhancement, anti-inflammatory effect and asthenia improving effect. The efficacy of the korean wild ginseng is similar to that of a cultivated ginseng, but the korean wild ginseng is more excellent with respect to the pharmacological effect.

Generally, a korean wild ginseng is divided into four categories in Korea, i.e. (“Cheonjong, Jijong, Injong, Jangnoi”). Specifically, Cheonjong (natural wild ginseng) naturally occurs from wild seeds; Jijong is propagated, for which animals or birds eat and excrete their seeds; Injong is cultured by human; and Jangnoi (wild simulated ginseng) is obtained by collecting the seed, seeding in the mountain, and artificially growing it in a wild state. A seed of the wild ginseng does not easily germinate, and even if the seed sprouts or germinates, it can survive just few years. Therefore, the wild ginseng is a rare plant which it is difficult to culture.

Recently, to solve the drawbacks such as a rareness of the wild ginseng and high value-added of the products, a method for mass-propagating the adventitious roots using the root tissue of a wild ginseng and the technique of the plant tissue culture had been known. Accordingly, the adventitious roots of a wild ginseng having uniform and excellent quality can be cultured in a large amount and a short period comparatively, which will be used for various foods or as a raw material. For example, in the case of preparing an alcoholic beverage, a cultivated ginseng, a Japanese apricot, gold was added and aged to improve its flavor, and a cultivated ginseng itself was contained in a beverage to enhance trust for the product with visual effect. However, a cosmetic composition comprising adventitious roots itself of a wild ginseng has not been published or disclosed yet.

Therefore, the inventors of the present invention have made extensive studies on preparation of a cosmetic composition, which stably comprises effective ingredients of a ginseng. They have found that the ingredient dip-extracted from the adventitious roots of a ginseng was uniformly maintained by adding a tissue-cultured adventitious roots itself of a ginseng to a cosmetic composition, and that user satisfaction was high in a sensory test, thereby completing the present invention.

### DISCLOSURE

### Technical Problem

Accordingly, it is an object of the present invention to provide a cosmetic composition comprising tissue-cultured adventitious roots itself of a ginseng, and a preparing method thereof, in which the effective ingredients are dip-extracted from the tissue-cultured adventitious roots itself of a ginseng and the content thereof were continuously maintained, and also a user recognizes visually the presence of the adventitious roots of a ginseng, thereby giving a user trust of the presence of the effective ingredient.

### Technical Solution

In order to achieve the object, the present invention provides a cosmetic composition comprising tissue-cultured adventitious roots itself of a ginseng and cosmetic ingredients.

The cosmetic composition of the invention comprises:
0.1 to 5 percent by weight of the tissue-cultured adventitious roots itself of the ginseng, based on the total weight of the cosmetic composition; and
95 to 99.9 percent by weight of the cosmetic ingredients, based on the total weight of the cosmetic composition.

The ginseng of the invention can be selected from the group consisting of a wild ginseng (“Sansam”), a cultivated ginseng (“Insam”), and a wild simulated ginseng (“Jangnoi”).

The cosmetic ingredients of the invention is selected from the group consisting of a solvent, an extender, an inorganic pigment, an organic pigment, an inorganic powder, an emulsion, a surfactant compatible a cosmetic composition, a silicone-contained surfactant, a thickener, a stabilizer, a preservative, an antioxidant, a flavoring agent, other cosmetic ingredients conventionally known in the art, and combinations thereof.

The composition is characterized in that, in the case of the composition comprises, as a solvent, 1 to 5 percent by weight of polyol based on the total weight of the composition, the ingredients that are effective for moisturization are dip-extracted from the blended tissue-cultured adventitious roots of a ginseng.

The composition is characterized in that, in the case of the composition comprises, as a solvent, 5 to 30 percent by weight of polyol based on the total weight of the composition, the ingredients that are effective for anti-wrinkles are dip-extracted from the blended tissue-cultured adventitious roots of a ginseng.

The composition is characterized in that, in the case of the composition comprises, as a solvent: 5 to 30 percent by weight of polyol based on the total weight of the composition; and 1 to 50 percent by weight of alcohol having 1 to 4 carbon atom based on the total weight of the composition, the ingredients that are effective for whitening are dip-extracted from the blended tissue-cultured adventitious roots of a ginseng.

The present invention further provides a preparing method for a cosmetic composition comprising tissue-cultured adventitious roots itself of a ginseng, comprising:
mixing conventional cosmetic ingredients; and
adding tissue-cultured adventitious roots itself of a ginseng to the cosmetic ingredient mixture.

A cosmetic composition of the present invention comprises ingredients that are effective for moisturization, anti-wrinkles, and whitening.

### ADVANTAGEOUS EFFECTS

The cosmetic composition of the present invention has advantages that the effective ingredients dip-extracted from tissue-cultured adventitious roots of a ginseng and the content thereof were continuously maintained, and also a user recognizes visually the presence of the adventitious roots itself of a ginseng, thereby improving a user's trust and satisfaction. Further, the preparing method for the cosmetic composition is a simple and inexpensive process without any additional step, such as extraction, isolation, and purification, and further has an advantage that the effective ingredients are dip-extracted from the adventitious roots itself of ginseng while not losing the effective ingredients.

### DESCRIPTION OF DRAWINGS

FIG. 1 is a photograph of the tissue-cultured adventitious roots of a korean wild ginseng.

FIG. 2 is a graph describing each preference for a cosmetics comprising tissue-cultured adventitious roots itself of a korean wild ginseng, and a cosmetics comprising the extract of a tissue-cultured adventitious roots of a wild ginseng.

FIG. 3 is a photograph of a cream and a serum, each comprising tissue-cultured adventitious roots itself of a korean wild ginseng, which correspond to Examples according to the present invention.

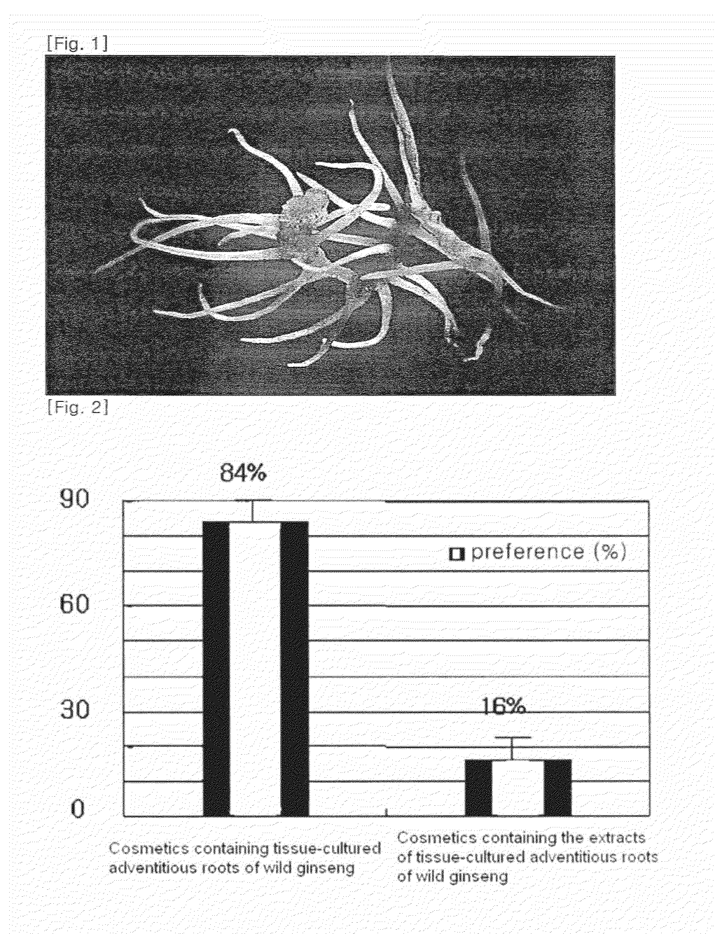

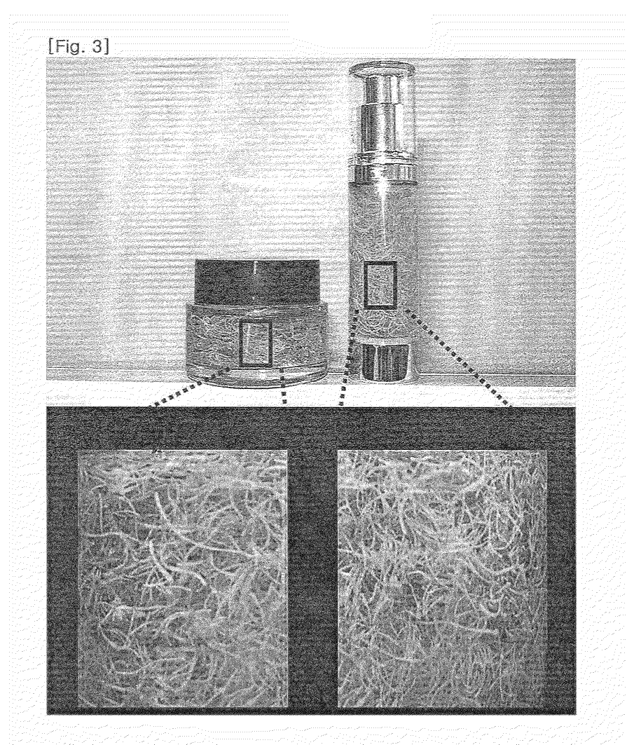

### MODE FOR INVENTION

Hereinbelow, the present invention will be described in detail.

In one embodiment of the invention, a cosmetic composition may be prepared by a method comprising:
mixing conventional cosmetic ingredients; and
adding tissue-cultured adventitious roots itself of a ginseng dried with hot air at 50 to 60° C. to the cosmetic ingredient mixture.

In another embodiment of the invention, the cosmetic ingredients may be mixed by a method conventionally known by a skilled person in the art.

In another embodiment of the invention, the tissue-cultured adventitious roots of a ginseng is characterized in that it is obtained by the tissue culture method disclosed in Korean patent No. 0353636.

Preferably, the cosmetic composition of the invention comprises: 0.1 to 5 percent by weight of the tissue-cultured adventitious roots of a ginseng, based on total weight of the cosmetic composition. If the content of the adventitious roots of a ginseng is less than 0.1 percent by weight, the content of effective ingredients dip-extracted becomes insufficient. If the content of the adventitious roots of a ginseng is more than 5 percent by weight, the states and properties of the product such as viscosity can be deteriorated, and stimulation on a user can be caused.

The cosmetic composition of the invention is characterized in that, in the case of the composition comprises polyol, as a solvent, preferably 1 to 5 percent by weight of 1,3-butyleneglycol based on total weight of the composition, ingredients that are effective for moisturization, preferably carbohydrates, are dip-extracted from the blended adventitious roots of a ginseng.

The cosmetic composition of the invention is characterized in that, in the case of the composition comprises polyol, as a solvent, preferably 5 to 30 percent by weight of 1,3-butyleneglycol based on total weight of the composition, ingredients that are effective for anti-wrinkles, preferably vitamins or saponins, are dip-extracted from the blended adventitious roots of a ginseng.

The cosmetic composition of the invention is characterized in that, in the case of the composition comprises polyol and alcohol having 1 to 4 carbon atom, as a solvent, preferably 5 to 30 percent by weight of 1,3-butyleneglycol and 1 to 50 percent by weight of ethanol based on total weight of the composition, ingredients that are effective for whitening, preferably flavonoids, are dip-extracted from the blended adventitious roots of a ginseng.

The cosmetic composition of the invention is found to contain 70% or more of the effective ingredients dip-extracted from the adventitious roots of a ginseng from 30 days after preparation, and reduction in the content of the effective ingredients is not observed until 180 days after aging. Thus, it can be used for a functional cosmetic composition.

If the tissue-cultured adventitious roots itself of a ginseng contained in the cosmetic composition of the invention is contaminated by various microorganisms, its color and phase may be changed. Accordingly, it can be an index that indicates contamination, and the cosmetic composition of the invention can be used with safety.

In the cosmetic composition of the present invention, the effective ingredients are naturally and continuously dip-extracted from the tissue-cultured adventitious roots of a ginseng, and maintained, and also allow a user to recognize visually the presence of the adventitious roots itself of a ginseng, thereby giving trust of the presence of the effective ingredient to the user, and improving user's satisfaction through its natural flavor.

The cosmetic composition of the invention can be used for various products, such as cosmetics, a facial cleanser, and a shampoo, for moisturizing, anti-wrinkle or whitening. The cosmetic composition can be added to various products, including a skin lotion, a skin softener, a skin toner, an astringent toner, a lotion, a milk lotion, a nutrient lotion, a massage cream, a nutrient cream, a hand cream, a foundation, a serum, a nutrient serum, a pack, a soap, a cleansing foam, a cleansing lotion, a cleansing cream, a body lotion or a body cleanser, but not limited thereto.

Hereinbelow, the present invention will be described in detail with reference to Examples and Experimental Examples.

Examples and Experimental Examples are provided only for the purpose of illustrating the present invention, and accordingly, it is not intended that the present invention is limited thereto.

### Reference Example

### Preparation of the Tissue-Cultured Adventitious Roots of a Korean Wild Ginseng

The tissue-cultured adventitious roots of a korean wild ginseng used in the present invention was purchased from CBN biotech (Korea). The adventitious roots of a korean wild ginseng were mass-propagated through tissue culture according to the method disclosed in Korean Patent No. 353636, and was dried with hot air at 50 to 60° C. before using.

### Example 1

### Preparation of Moisturizing Serum Comprising Tissue-Cultured Adventitious Roots Itself of a Korean Wild Ginseng

A moisturizing serum comprising tissue-cultured adventitious roots itself of a korean wild ginseng was prepared according to the composition as shown in the following Table 1.

TABLE 1

| | Ingredients | Content (wt %) |
|---|---|---|
| A | 1,3-Butyleneglycol | 1.0 |
| | Glycerine | 1.0 |
| | Carbomer | 0.2 |
| | Glyceryl methacrylate | 0.35 |
| | Paraben | 0.20 |
| | Purified water | up to 100 |
| B | Potassium hydroxide | 0.06 |
| C | PEG-6-hydrogenated castor oil | 1.0 |
| | Flavoring agent | Proper amount |
| D | Tissue-cultured adventitious roots of | 2.0 |
| | wild ginseng in Reference Example 1 | |
| | Total | 100.00 |

A was sufficiently dispersed and moisturized to be in a uniform gel state, and B was added thereto for neutralization. After C was added to (A+B) to solubilize with uniform stirring, D was added thereto at room temperature. The mixture was stirred in order to disperse uniformly, and then was put into a container for commercialization. When the serum prepared by the above method was applied to skin, its moisturizing effect was continuously maintained.

### Example 2

### Preparation of Moisturizing Cream Comprising Tissue-Cultured Adventitious Roots Itself of a Korean Wild Ginseng

A moisturizing cream comprising tissue-cultured adventitious roots itself of a korean wild ginseng was prepared according to the composition as shown in the following Table 2.

TABLE 2

| | Ingredients | Content (wt %) |
|---|---|---|
| A | 1,3-Butyleneglycol | 1.0 |
| | Glycerine | 1.0 |
| | Carbomer | 0.5 |
| | Glyceryl methacrylate | 0.35 |
| | Paraben | 0.20 |
| | Purified water | up to 100 |
| B | Potassium hydroxide | 0.15 |
| C | PEG-6-hydrogenated castor oil | 1.0 |
| | Flavoring agent | Proper amount |
| D | Tissue-cultured adventitious roots of | 2.0 |
| | wild ginseng in Reference Example 1 | |
| | Total | 100.00 |

The cream in Example 2 was prepared in the same manner as in Example 1. When the cream prepared by the above method was applied to skin, its moisturizing effect was continuously maintained.

### Example 3

### Preparation of Anti-Wrinkle Serum Comprising Tissue-Cultured Adventitious Roots Itself of a Korean Wild Ginseng

An anti-wrinkle serum comprising tissue-cultured adventitious roots itself of a korean wild ginseng was prepared according to the composition as shown in the following Table 3.

TABLE 3

| | Ingredients | Content (wt %) |
|---|---|---|
| A | 1,3-Butyleneglycol | 5.0 |
| | Glycerine | 5.0 |
| | Carbomer | 0.2 |
| | Glyceryl methacrylate | 0.35 |
| | Paraben | 0.20 |
| | Purified water | up to 100 |
| B | Potassium hydroxide | 0.06 |
| C | PEG-6-hydrogenated castor oil | 1.0 |
| | Flavoring agent | Proper amount |
| D | Tissue-cultured adventitious roots of | 2.0 |
| | wild ginseng in Reference Example 1 | |
| | Total | 100.00 |

The serum in Example 3 was prepared in the same manner as in Example 1. When the serum prepared by the above method was applied to skin, its anti-wrinkle effect was continuously maintained.

### Example 4

### Preparation of Anti-Wrinkle Cream Comprising Tissue-Cultured Adventitious Roots Itself of a Korean Wild Ginseng

An anti-wrinkle cream comprising tissue-cultured adventitious roots itself of a korean wild ginseng was prepared according to the composition as shown in the following Table 4.

TABLE 4

| | Ingredients | Content (wt %) |
|---|---|---|
| A | 1,3-Butyleneglycol | 5.0 |
| | Glycerine | 5.0 |
| | Carbomer | 0.5 |
| | Glyceryl methacrylate | 0.35 |
| | Paraben | 0.20 |
| | Purified water | up to 100 |
| B | Potassium hydroxide | 0.15 |
| C | PEG-6-hydrogenated castor oil | 1.0 |
| | Flavoring agent | Proper amount |
| D | Tissue-cultured adventitious roots of | 2.0 |
| | wild ginseng in Reference Example 1 | |
| | Total | 100.00 |

The cream in Example 4 was prepared in the same manner as in Example 1. When the cream prepared by the above method was applied to skin, its anti-wrinkle effect was continuously maintained.

### Example 5

### Preparation of Whitening Serum Comprising Tissue-Cultured Adventitious Roots Itself of a Korean Wild Ginseng

A whitening serum comprising tissue-cultured adventitious roots itself of a korean wild ginseng was prepared according to the composition as shown in the following Table 5.

TABLE 5

| | Ingredients | Content (wt %) |
|---|---|---|
| A | 1,3-Butyleneglycol | 5.0 |
| | Glycerine | 5.0 |
| | Ethanol | 10.0 |
| | Carbomer | 0.2 |
| | Glyceryl methacrylate | 0.35 |
| | Paraben | 0.20 |
| | Purified water | up to 100 |
| B | Potassium hydroxide | 0.06 |
| C | PEG-60-hydrogenated castor oil | 1.0 |
| | Flavoring agent | Proper amount |
| D | Tissue-cultured adventitious roots of | 2.0 |
| | wild ginseng in Reference Example 1 | |
| | Total | 100.00 |

The serum in Example 5 was prepared in the same manner as in Example 1. When the serum prepared by the above method was applied to skin, its whitening effect was continuously maintained.

### Example 6

### Preparation of Whitening Cream Comprising Tissue-Cultured Adventitious Roots Itself of a Korean Wild Ginseng

A whitening cream comprising the tissue-cultured adventitious roots itself of a korean wild ginseng was prepared according to the composition as shown in the following Table 6.

TABLE 6

| | Ingredients | Content (wt %) |
|---|---|---|
| A | 1,3-Butyleneglycol | 5.0 |
| | Glycerine | 5.0 |
| | Ethanol | 10.0 |
| | Carbomer | 0.5 |
| | Glyceryl methacrylate | 0.35 |
| | Paraben | 0.20 |
| | Purified water | up to 100 |
| B | Potassium hydroxide | 0.15 |
| C | PEG-60-hydrogenated castor oil | 1.0 |
| | Flavoring agent | Proper amount |
| D | Tissue-cultured adventitious roots of | 2.0 |
| | wild ginseng in Reference Example 1 | |
| | Total | 100.00 |

The cream in Example 6 was prepared in the same manner as in Example 1. When the cream prepared by the above method was applied to skin, its whitening effect was continuously maintained.

### Comparative Example 1 to 6

### Preparation of Cosmetic Composition not Comprising Tissue-Cultured Adventitious Roots Itself of a Korean Wild Ginseng

A cosmetic composition not comprising tissue-cultured adventitious roots itself of a korean wild ginseng was prepared with the same ingredients as in Example 1 to 6, except an tissue-cultured adventitious roots of a korean wild ginseng.

### Experimental Example 1

### Efficacy Test of Cosmetic Composition According to Present Invention

### 1-1. Test for Moisturizing Effect

To confirm the moisturizing effect of the cosmetic composition according to the present invention, the hydration level in skin was measured. Each cosmetic compositions prepared according to the formulations in Examples 1 and 2, and Comparative Examples 1 and 2 was applied to twenty female subjects, who were 20 to 50 years old. The hydration level in skin was measured before and after applying each cosmetic composition as following.

The hydration level in skin was measured by means of a corneometer (CM 825, Courage & Khazaka, Germany), which is a conventional skin hydration measurement device using capacitance measurement of a dielectric medium. Each sample of 2.0 mg/cm² was uniformly applied to one upper arm of subjects, who were waiting for 1 hour before test. Each sample was not applied to the other upper arm of subjects, as a control group. The hydration level in skin was measured immediately before and after applying each sample. Further, the moisture content in skin was measured in 1 hour, 3 hours, and 5 hours, after applying each sample, respectively.

TABLE 7

| | | Result | |
|---|---|---|---|
| | | | Corneometric |
| | | Corneometric | value |
| | Time | value | (non-applied |
| Sample | (hr) | (applied portion) | portion) |
| comparative Example 1 | 0 | 80 | 79 |
| | 1 | 99 | 80 |
| | 3 | 95 | 81 |
| | 5 | 85 | 79 |
| Example 1 (in 30 days after | 0 | 81 | 81 |
| preparation) | 1 | 110 | 82 |
| | 3 | 106 | 80 |
| | 5 | 102 | 81 |
| Example 1 (in 60 days after | 0 | 83 | 75 |
| preparation) | 1 | 112 | 80 |
| | 3 | 105 | 78 |
| | 5 | 99 | 82 |
| Example 1 (in 90 days after | 0 | 82 | 79 |
| preparation) | 1 | 110 | 79 |
| | 3 | 104 | 81 |
| | 5 | 98 | 80 |
| Example 1 (in 120 days after | 0 | 85 | 82 |
| preparation) | 1 | 109 | 80 |
| | 3 | 100 | 79 |
| | 5 | 95 | 82 |
| Example 1 (in 150 days after | 0 | 83 | 81 |
| preparation) | 1 | 109 | 83 |
| | 3 | 101 | 80 |
| | 5 | 94 | 81 |
| Example 1 (in 180 days after | 0 | 87 | 79 |
| preparation) | 1 | 114 | 81 |
| | 3 | 108 | 82 |
| | 5 | 99 | 80 |

TABLE 8

| | | Result | |
|---|---|---|---|
| | | | Corneometric |
| | | Corneometric | value |
| | Time | value | (non-applied |
| Sample | (hr) | (applied portion) | portion) |
| Comparative Example 2 | 0 | 88 | 80 |
| | 1 | 102 | 80 |
| | 3 | 95 | 78 |
| | 5 | 89 | 79 |
| Example 2 (in 30 days after | 0 | 87 | 80 |
| preparation) | 1 | 116 | 79 |
| | 3 | 106 | 81 |
| | 5 | 93 | 82 |
| Example 2 (in 60 days after | 0 | 84 | 79 |
| preparation) | 1 | 114 | 79 |
| | 3 | 103 | 80 |
| | 5 | 97 | 81 |
| Example 2 (in 90 days after | 0 | 83 | 81 |
| preparation) | 1 | 114 | 80 |
| | 3 | 105 | 81 |
| | 5 | 94 | 80 |
| Example 2 (in 120 days after | 0 | 85 | 79 |
| preparation) | 1 | 110 | 81 |
| | 3 | 99 | 80 |
| | 5 | 92 | 81 |
| Example 2 (in 150 days after | 0 | 83 | 81 |
| preparation) | 1 | 113 | 79 |
| | 3 | 101 | 79 |
| | 5 | 92 | 82 |
| Example 2 (in 180 days after | 0 | 84 | 82 |
| preparation) | 1 | 116 | 79 |
| | 3 | 102 | 80 |
| | 5 | 96 | 81 |

As shown in Tables 7 and 8, there is a significant difference in the hydration level between the portions which were applied with the cosmetic compositions of Example 1 or 2 and those which were not applied.

The hydration level in the portions which were applied with the cosmetic compositions of Example 1 or 2 in more than 30 days after preparation was excellent, compared with those in the portions which were applied with the cosmetic compositions of Comparative Example 1 or 2. It was also found that the hydration level in the portions which were applied with the cosmetic compositions of Example 1 or 2 in 180 days after preparation were excellently maintained.

### 1-2. Test for Effect of Promoting Collagen Biosynthesis

To confirm the anti-wrinkle effect of the cosmetic composition according to the present invention, the effect of promoting collagen biosynthesis was measured using fibroblasts. Mouse fibroblast (3T3-L1, KCLB 10092.1, Korean Cell Line Bank) were cultured using DMEM medium (Dulbecco's Modified Eagle's Medium, Invitrogen, USA) containing 10% (v/v) FBS (fetal bovine serum, BioWhittaker, MD, USA) and 1% (v/v) penicillin-streptomycin (BioWhittaker, MD, USA) under the condition of 5% CO₂ and 37° C. After cell culture, the cells were collected with treating trypsin. The collected cells were seeded in a 24-well plate at a concentration of 1×10⁵ cells/ml/well, followed by culturing for 24 hours. After cell culture, each cosmetic compositions prepared according to the formulations in Example 3 and 4, and Comparative Examples 3 and 4 were diluted to 0.01%. The cells were treated with the diluted cosmetic compositions and cultured for 24 hours, followed by using for the following experiment.

In order to confirm whether collagen biosynthesis was enhanced in the fibroblast or not, the supernatant of the cultured cells was collected to determine the amount of collagen.

Specifically, 1 ml of a dye reagent (prepared by dissolving 0.1 g of a Sirius red reagent (BDH, UK) in 100 ml of saturated picric acid) was added to 100 μl of the supernatant. The mixture was subjected to reaction under slow stirring at room temperature for 30 minutes. Sirius red is an anionic pigment, which specifically binds to collagen. After 30 minutes reaction, a collagen-pigment complex was precipitated by a centrifuge at over 5000×g for 20 minutes. 0.5 M of NaOH was added to the precipitate to dissolve at room temperature for 5 minutes. Absorbance of the solution was measured using a micro well plate reader at 540 nm. The result was shown in Tables 9 and 10. The results were compared with the absorbance values of the non-test group to evaluate the effect of promoting collagen biosynthesis.

TABLE 9

| | Ratio of collagen |
|---|---|
| Sample | biosynthesis (%) |
| Comparative Example 3 | 101% |
| Example 3 (in 30 days after preparation) | 108% |
| Example 3 (in 60 days after preparation) | 110% |
| Example 3 (in 90 days after preparation) | 118% |
| Example 3 (in 120 days after preparation) | 121% |
| Example 3 (in 150 days after preparation) | 118% |
| Example 3 (in 180 days after preparation) | 118% |

TABLE 10

| | Ratio of collagen |
|---|---|
| Sample | biosynthesis (%) |
| Comparative Example 4 | 103% |
| Example 4 (in 30 days after preparation) | 112% |
| Example 4 (in 60 days after preparation) | 120% |
| Example 4 (in 90 days after preparation) | 121% |
| Example 4 (in 120 days after preparation) | 119% |
| Example 4 (in 150 days after preparation) | 119% |
| Example 4 (in 180 days after preparation) | 118% |

As shown in Tables 9 and 10, there is a significant difference in a ratio of collagen biosynthesis between the fibroblast treated with the cosmetic compositions of Example 3 or 4 and the fibroblast treated with the cosmetic compositions of Comparative Example 3 or 4.

A ratio of collagen biosynthesis increased in, from the group which was treated with a cosmetic composition in 30 days after preparation, to the group which was treated with a cosmetic composition in 180 days after preparation. It was found that the ratio of collagen biosynthesis of the groups treated with the cosmetic compositions maintained 1.2 times than that of a control group.

### 1-3. Test for Effect of Inhibiting Tyrosinase Activity

To confirm the whitening effect of the cosmetic composition according to the present invention, the effect of inhibiting tyrosinase activity was measured. A mushroom tyrosinase (T-3824, 1530U/mg, Sigma) was dissolved in a phosphate buffer (pH 6.5) to the concentration of 1000 U/ml to prepare a tyrosinase enzyme solution. L-tyrosine (45160-0410, Junsei chemical co. Ltd) was dissolved in a phosphate buffer (pH 6.5) to the concentration of 1.5 mM to prepare a substrate solution.

Each cosmetic compositions prepared according to the formulations in Example 5 and 6, and Comparative Example 5 and 6 was added to 20 g of purified water and stirred to be a total volume of 500 ml. 1 N of hydrochloric acid was added thereto to adjust the pH to 2.0. The solution was placed in a separatory funnel. The equal amount of chloroform was added thereto. The solution was vigorously shaken and left to separate an aqueous layer. After the operation was repeated twice times, the obtained aqueous layer was concentrated under reduced pressure at 60° C. 1 N of sodium hydroxide was added thereto to adjust the pH to 6.5. Further, purified water was added thereto to be a volume of 20 ml. The solution was filtered and a phosphate buffer (pH 6.5) was added to 5 ml of filtrate to be a volume of 10 ml as a test solution. 10 μl of the tyrosinase enzyme solution was added to 170 μl of the test solution, and left at 37° C. for 10 minutes. 20 μl of the substrate solution was added thereto. The reaction was carried out at 37° C. for 10 minutes, and immediately left in ice for 5 minutes. Absorbance was measured using an ELISA reader (UV max kinetic microplate reader 01414, Molecular Devices, USA) at 490 nm. The absorbance measured was substituted into Equation 1 to calculate the inhibition ratio of tyrosinase activity. The result was shown in the following Table 11 and 12. A phosphate buffer (pH 6.5) instead of the test solution was added to prepare a blank solution. Further, a phosphate buffer (pH 6.5) instead of the substrate solution was added to prepare a calibration solution.

Inhibition ratio of tyrosinase activity(%)=100−[(A−A′)/(B−B′)]×100 [Equation 1]

A: Absorbance after reaction of test solution
B: Absorbance after reaction of blank solution
A′: Calibration solution of test solution
B′: Calibration solution of blank solution

TABLE 11

| | Inhibition ratio of |
|---|---|
| Sample | tyrosinase activity (%) |
| Comparative Example 5 | 23% |
| Example 5 (in 30 days after preparation) | 46% |
| Example 5 (in 60 days after preparation) | 54% |
| Example 5 (in 90 days after preparation) | 55% |
| Example 5 (in 120 days after preparation) | 63% |
| Example 5 (in 150 days after preparation) | 65% |
| Example 5 (in 180 days after preparation) | 63% |

TABLE 12

| | Inhibition ratio of |
|---|---|
| Sample | tyrosinase activity (%) |
| Comparative Example 6 | 32% |
| Example 6 (in 30 days after preparation) | 56% |
| Example 6 (in 60 days after preparation) | 59% |
| Example 6 (in 90 days after preparation) | 64% |
| Example 6 (in 120 days after preparation) | 64% |
| Example 6 (in 150 days after preparation) | 71% |
| Example 6 (in 180 days after preparation) | 70% |

As shown in Tables 11 and 12, it was found that when the cosmetic compositions in Examples 5 and 6 according to the present invention were aged for 30 days to 180 days after preparation, effective ingredients for whitening were dip-extracted from the adventitious roots of a korean wild ginseng to increase the inhibition ratio of tyrosinase activity. Therefore, it was found that the activity of the effective ingredients for whitening was maintained.

### Experimental Example 2

### Preference Survey for Cosmetics Comprising Tissue-Cultured Adventitious Roots Itself of a Korean Wild Ginseng

A preference survey for the formulations in Example 1 and 2, and Comparative formulations (serum and cream comprising an extract of the adventitious roots of a wild ginseng) was performed by 100 female subjects, who were 20 to 50 years old. The results of preference survey were shown in FIG. 2.

As shown in FIG. 2, the presence of the adventitious roots itself of a ginseng can be visually recognized, and as a result, it can be found that 84 females of 100 females prefer the formulation comprising the adventitious roots itself of a wild ginseng to the formulation comprising the extract of the adventitious roots of a wild ginseng.

1. A cosmetic composition comprising tissue-cultured adventitious roots itself of a ginseng and cosmetic ingredients.

2. The cosmetic composition according to claim 1, wherein said composition comprises:
0.1 to 5 percent by weight of the tissue-cultured adventitious roots itself of the ginseng, based on the total weight of the cosmetic composition; and
95 to 99.9 percent by weight of the cosmetic ingredients, based on the total weight of the cosmetic composition.

3. The cosmetic composition according to claim 1, wherein the ginseng is selected from the group consisting of a wild ginseng, a cultivated ginseng and a wild simulated ginseng.

4. The cosmetic composition according to claim 1, wherein the cosmetic ingredients is selected from the group consisting of a solvent, an extender, an inorganic pigment, an organic pigment, an inorganic powder, an emulsion, a surfactant compatible a cosmetic composition, a silicone-contained surfactant, a thickener, a stabilizer, a preservative, an antioxidant, a flavoring agent and combinations thereof.

5. The cosmetic composition according to claim 1, wherein said composition comprises, as a solvent, 1 to 5 percent by weight of polyol based on the total weight of the composition, and wherein the composition has a moisturizing effect.

6. The cosmetic composition according to claim 1, wherein said composition comprises, as a solvent, 5 to 30 percent by weight of polyol based on the total weight of the composition, and wherein the composition has an anti-wrinkle effect.

7. The cosmetic composition according to claim 1, wherein said composition comprises as a solvent: 5 to 30 percent by weight of polyol; and 1 to 50 percent by weight of alcohol having 1 to 4 carbon atom, said percentages are based on the total weight of the composition, and wherein the composition has a whitening effect.

8. A preparing method for a cosmetic composition comprising tissue-cultured adventitious roots itself of a ginseng, comprising:
mixing conventional cosmetic ingredients; and
adding tissue-cultured adventitious roots itself of a ginseng to the cosmetic ingredient mixture.

9. The preparing method according to claim 8, wherein the cosmetic composition comprises ingredients that are effective for moisturization, anti-wrinkles, or whitening.
